(19) United States
(12) Patent Application Publication
(10) Pub. No.: US 2021/0023494 A1
(43) Pub. Date: Jan. 28, 2021

(54) FILTER ELEMENT HAVING A FUNCTIONAL SPACE AND FILTER ARRANGEMENT HAVING SUCH A FILTER ELEMENT, AND METHOD FOR CONTROLLING A FILTER SYSTEM

(71) Applicant: Carl Freudenberg KG, Weinheim (DE)

(72) Inventors: Alexander Oelsner, Mannheim (DE); Volker Braeunling, Heppenheim (DE); Ulrich Stahl, Laudenbach (DE)

(21) Appl. No.: 16/928,014

(22) Filed: Jul. 14, 2020

(30) Foreign Application Priority Data
Jul. 24, 2019 (EP) 19 188 126.7

(51) Int. Cl. B01D 46/42; B01D 46/00; B60H 3/00; B60H 3/06

(52) U.S. Cl. CPC B01D 46/42; B01D 46/0038; B01D 46/52; B60H 3/0014; B60H 3/0658; B01D 46/0086

(57) ABSTRACT

A filter element includes: a bellows including a pleated filter medium and edge strips bounding the bellows; and a functional space that adjoins the bellows and is arranged within the edge strips, having an interface between the bellows and functional space. The interface is provided with at least one cutout for receiving sensor elements and/or fragrancing elements.

### CROSS-REFERENCE TO PRIOR APPLICATION

Priority is claimed to European Patent Application No. EP 19 188 126.7, filed on Jul. 24, 2019, the entire disclosure of which is hereby incorporated by reference herein.

### FIELD

The invention relates to a filter element.

### BACKGROUND

A filter element with an integrated container is known from WO 2007/093272 A1. Thereby, the container can serve to receive a fragrancing element. With such fragrancing elements, a certain fragrance can be added to an air stream in the filter element.

Filter elements with fragrancing elements are also disclosed in US 2007/0261376 A1 and EP 2 868 987 A1.

Filter elements which have sensors are known from DE 10 2009 040 707 A1, WO 2016/016085 A1 and US 2003/0052791 A1. Thereby, the sensors are mounted directly on the filter medium.

### SUMMARY

In an embodiment, the present invention provides a filter element, comprising: a bellows comprising a pleated filter medium and edge strips bounding the bellows; and a functional space that adjoins the bellows and is arranged within the edge strips, having an interface between the bellows and functional space, wherein the interface is provided with at least one cutout configured to receive sensor elements and/or fragrancing elements.

### BRIEF DESCRIPTION OF THE DRAWINGS

The present invention will be described in even greater detail below based on the exemplary figures. The invention is not limited to the exemplary embodiments. Other features and advantages of various embodiments of the present invention will become apparent by reading the following detailed description with reference to the attached drawings which illustrate the following:

FIG. 1 shows a first embodiment variant of a filter element according to the invention;

FIG. 2 shows a second embodiment variant of a filter element according to the invention;

FIG. 3a shows a section through the filter element;

FIG. 3b shows a view of the separating surface; and

FIG. 4 shows a top view of a filter arrangement.

### DETAILED DESCRIPTION

In an embodiment, the present invention provides a filter element which has a functional space which makes it possible in a simple manner to provide at least one sensor element and/or at least one fragrancing element and a simple replacement of the fragrancing elements.

In an embodiment, the present invention provides a filter arrangement which makes possible a simple replacement of a filter element, in particular if the filter element includes a reusable sensor element.

In an embodiment, the present invention provides a filter element having a functional space as described herein.

According to the invention, it has been found advantageous to provide the functional space within the edge strips of the filter element, so that the filter element has no greater volume with its additional functionality and can be used in existing filter arrangements.

The filter element according to the invention has a bellows made of a pleated filter medium with a multiplicity of folding edges and folding surfaces and has edge strips for bounding the filter medium, so that each edge strip forms a wall of the filter element. The filter element has a functional space which is firmly connected to the filter element, adjoins the bellows and is arranged within the edge strips. This means that the functional space—in a top view of the filter element with its folding surfaces of the bellows—is adjacent to the bellows. An interface is arranged between bellows and functional space and separates the bellows from the functional space. The interface is advantageously provided with at least one cutout, for example a recess or a hole for receiving at least one sensor element and/or at least one fragrancing element in the region of the bellows. Thereby, the sensor elements can serve for measuring particles, gases, fragrances, etc. in an air stream through the filter element. In particular, the sensor elements and/or fragrancing elements cannot only be accommodated in the filter element, but are actually present and part of the filter element.

The cutouts are configured in such a way that a sensor head of the sensor element or the fragrancing element can be positioned in the region of the bellows. In order to provide sufficient space for the sensor elements and/or fragrancing elements, the fold spacing and/or the fold height of the bellows may possibly be changed locally in such a way that more space is created for sensor elements or fragrancing elements. Any fragrancing elements present are preferably arranged on the clean gas side of the bellows, since the fragrances can thus be discharged directly into the cleaned air.

The filter element according to the invention is advantageously of particularly compact design and a simple equipping with fragrancing elements or sensor elements is made possible. The integration of the functional space into the filter element also ensures that the number of possible bypasses and thus the risk of missing air is reduced.

In an advantageous development of the filter element according to the invention, at least two cutouts are provided in the interface for receiving at least one sensor element in one cutout and at least one fragrancing element in another cutout. In this development, both a fragrancing of the air stream through the filter element and a measurement of certain properties of the air stream can advantageously take place. All elements required for this purpose are integrated into the filter element.

In a possible development of the filter element according to the invention, at least two cutouts are provided in the interface for receiving two sensor elements, wherein one cutout and thus also one sensor element are in each case positioned on the clean gas side and one cutout and thus also one sensor element are positioned on the raw gas side of the bellows. Thanks to such a measurement of incoming air and outgoing air, statements regarding air quality, cleaning performance or filtration performance, service life of the filter, required filter replacement, etc. are possible.

In an alternative development or even a further development of the filter element according to the invention, at least one cutout is provided in the interface for accommodating at least one fragrancing element, wherein the at least one cutout is positioned on the clean gas side of the bellows, so that direct fragrancing of the cleaned air stream with fragrance is made possible.

In an alternative development or also further development of the filter element according to the invention, at least one cutout is provided in the interface for accommodating at least one fragrancing element, wherein the at least one cutout is positioned on the raw gas side of the bellows and the fragrancing element has a metering agent with an oxidizing effect. A metering agent with a cleaning effect for the filter medium of the bellows can be provided by the fragrancing element on the raw gas side.

In an alternative development or even a further development of the filter element according to the invention, at least one cutout is provided in the interface for accommodating at least one fragrancing element, wherein the at least one cutout is positioned on the clean gas side of the bellows and the fragrancing element has a metering agent with an oxidizing effect. Due to the fragrancing element on the clean gas side, a metering agent with a cleaning effect for components downstream of the filter element, such as for example, air ducts, heat exchangers, control valves, etc. can be provided.

When such developments are combined, a filter element is obtained in which at least two cutouts are provided in the interface for accommodating two fragrancing elements, wherein one fragrancing element is in each case positioned on the clean gas side and one fragrancing element is positioned on the raw gas side of the bellows. Direct fragrancing of the cleaned air stream with fragrance is made possible by the fragrancing element on the clean gas side. A metering agent for the filter medium of the bellows can be provided by the fragrancing element on the raw gas side. A metering agent with a cleaning effect for components downstream of the filter element can be provided by means of a further fragrancing element on the clean gas side.

Surprisingly, it has been found that a metering agent with an oxidizing effect, such as a silicate-enriched sodium hypochlorite solution, can have a cleaning effect on the filter medium or on downstream components with respect to microorganisms and viruses, which leads to an extension of the filter life of the filter element or to a prevention of a “contamination” of the downstream components.

In an advantageous development of the filter element according to the invention, the functional space is open only to one side and is thus not closed off completely but is accessible. In particular, the functional space is open towards the raw gas side. The functional space can, for example, be in the shape of a cuboid which is open to one side.

In a first possible embodiment variant of the filter element, the functional space extends parallel to the folding edges of the bellows and the folding surfaces of the bellows are penetrated by sensor elements and/or fragrancing elements that may be present. Corresponding cutouts are also provided in the folding surfaces of the bellows for this purpose. The interface of the filter element can either be formed from the filter medium or take the form of an edge strip.

According to the second embodiment variant, the functional space is positioned adjacent to the end faces of the fold edges of the bellows and the interface takes the form of an edge strip. In this embodiment variant, sensor elements and/or fragrancing elements that may be present can lie between or above folding surfaces, so that the folding surfaces of the bellows remain whole.

In an advantageous development, a sensor component can be arranged in the functional space, wherein the sensor component and the at least one sensor element in each case form one or more sensors. The sensor element can thus essentially include the measuring head and the sensor component the control and power supply of the sensor head. The sensor element and the sensor component can either be separate elements. However, the sensor element and the sensor component can also be combined to form one component.

In an advantageous development, a transmitter is additionally arranged in the functional space, which transmitter is connected to the sensor in terms of data transmission technology for the data transmission of measurement data of the sensor. The measurement data of the sensor can thus be transmitted to a receiver, which is mounted, for example, in a housing of the filter element, or in the system or machine in which the filter element is being used. In particular, the transmitter can take the form of an NFC transmitter, of a near-field communication transmitter, for example using RFID or Bluetooth.

The invention also relates to a filter arrangement having a frame and a filter element as described above, wherein the filter element is accommodated in the frame. Frame is understood here to also mean a housing. In such a development of a filter arrangement, a functional space is advantageously created within the filter element. The frame need not be modified and no larger space is needed for the filter element. Nor is any additional sealing of the functional space required for leakage prevention.

The invention also relates to the use of such a filter arrangement for air filtration in a mobile device, in particular in a motor vehicle or a commercial vehicle.

The invention also relates to a method for controlling a filter system having a filter arrangement as described above. The filter system further comprises a control device for controlling the filter element, wherein the control device is connected in terms of data transmission technology to at least one sensor in the filter element, and controlling of the filter element takes place as a function of measured values of the sensor. For example, the control of the filter element can include the control of the volumetric flow, an ionization of the incoming air and a regulation of the circulating air/fresh air component.

The invention will now be explained in more detail using the accompanying figures. Corresponding elements and components are provided with the same reference symbols in the figures. For the sake of better clarity of the figures, a presentation that is true to scale has been dispensed with.

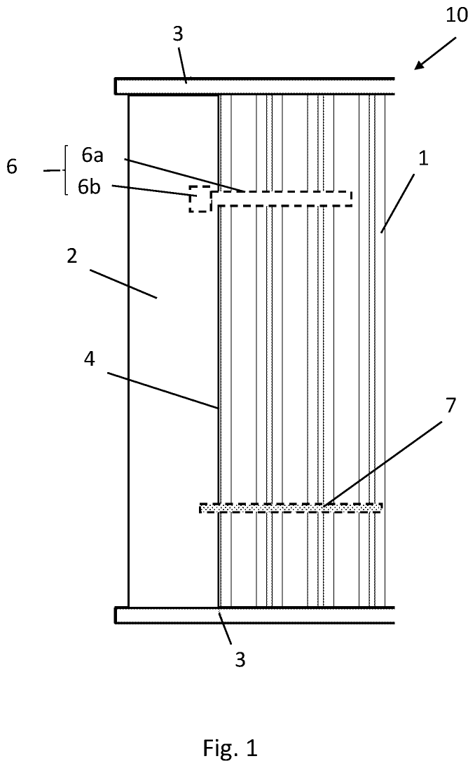

FIG. 1 shows a filter element 10 in a top view from the raw gas side. The filter element 10 has a bellows 1 made of pleated filter medium and a functional space 2 directly adjacent to the bellows 1. The functional space 2 extends parallel to the folding edges of the bellows 1. The bellows 1 and the functional space 2 are separated from each other by an interface 4. Edge strips 3 are provided for stabilizing and sealing the bellows 1. The functional space 2 is located inside the edge strips 3 and is firmly connected to the bellows 1 and thus forms part of the filter element 10. Cutouts 5a, 5b are provided in the interface 4, through which sensor elements 6a and fragrancing elements 7 can be introduced and inserted in such a way that they are located in the region of the bellows 1. Any sensor elements 6a and fragrancing elements 7 that may be present pass through the folding surfaces of the bellows 1 for which purpose cutouts are likewise provided in the folding surfaces (not shown). In the representation in FIG. 1, such a sensor element 6a and such a fragrancing element 7 are indicated by dashed lines. A sensor component 6b which is located in the functional space 2 or projects into this is integrally connected to the sensor element 6a. The sensor element 6a and the sensor component 6b together form a sensor 6.

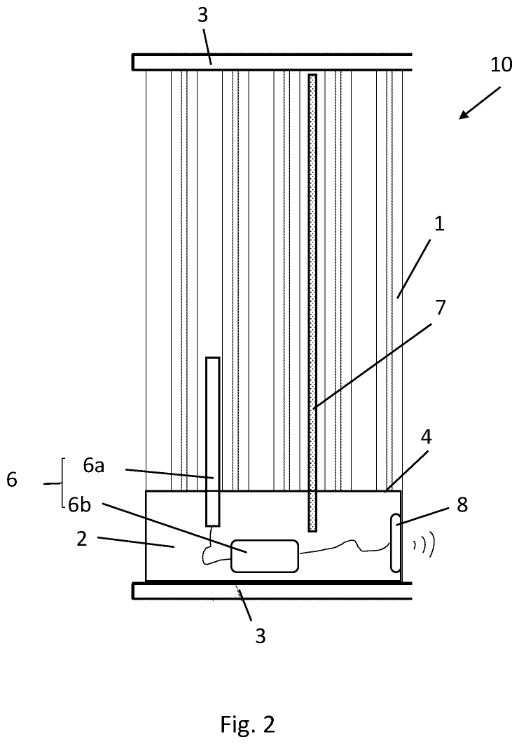

FIG. 2 shows a second alternative arrangement of the functional space 2 relative to the bellows 1. The functional space 2 adjoins the bellows 1 at one side, specifically where the end faces of the folding edges of the bellows 1 are located. The functional space 2 is separated from the bellows 1 by an interface 4. In this case as well, the functional space 2 lies within the boundaries of the edge strips 3. The interface 4 can also take the form of an edge strip 3. In the top view of FIG. 2, a sensor 6 and a fragrancing element 7 can be seen. The sensor 6 is connected to a transmitter 8 in terms of data transmission technology, which transmitter is able to transmit measurement data of the sensor 6 to a receiver (not shown).

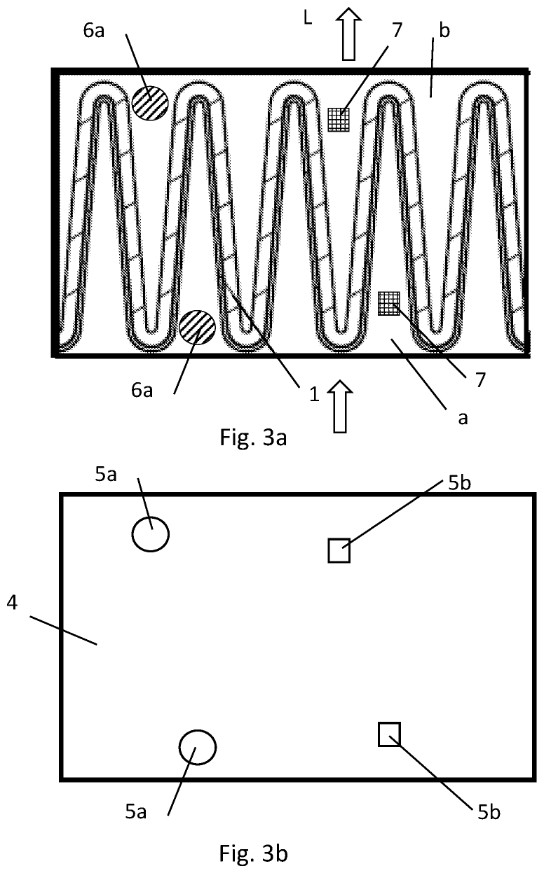

While each of FIGS. 1 and 2 showed only one fragrancing element and one sensor element, the filter element 10 can also be equipped with a plurality of sensor elements 6a and a plurality of fragrancing elements 7. This is illustrated in FIGS. 3a and 3b.

FIG. 3a shows a sectional view through the filter element 10 in the region of its bellows 1. An air stream L flows through the bellows 1. The air is still unfiltered on the raw gas side a, while on the clean gas side b the air is already filtered. The filter element 10 has two sensor elements 6a and two fragrancing elements 7. A first sensor element 6a is arranged on the raw gas side a and a second sensor element 6a is arranged on the clean gas side b. This makes it possible to measure the air before and after filtering by the filter element 10. While the fragrancing element 7 on the clean gas side element (at b) serves for a direct fragrancing of the air stream L, the metering substances positioned on the raw gas side (at a) that have an oxidizing effect and are released by the fragrancing element 7 serve to clean the filter medium of the bellows 1, which leads to an extension of the filter life.

FIG. 3b shows the associated interface 4 which has two cutouts 5a for the sensor elements 6a and two cutouts 5b for the fragrancing elements 7. The cutouts 5a and 5b here have a circular or square basic shape. However, other shapes are also conceivable for the cutouts 5a, 5b, for example also non-point-symmetrical shapes which allow sensor elements 6a and fragrancing elements 7 to be inserted only with a predetermined orientation.

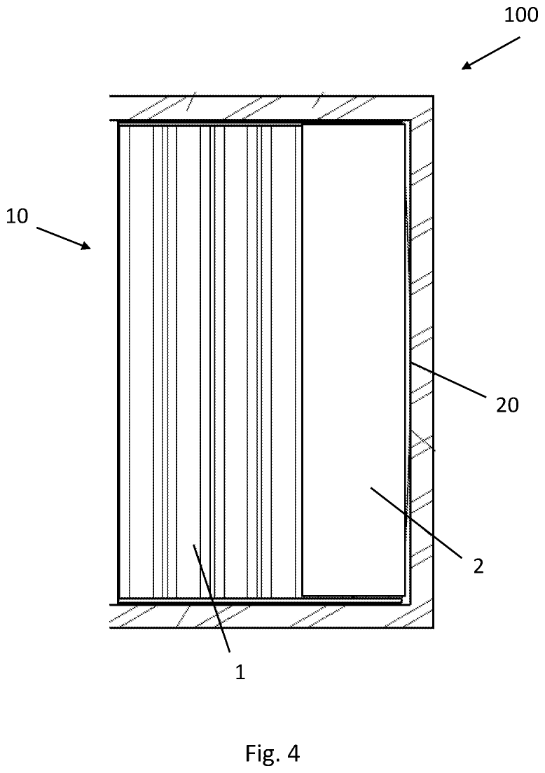

FIG. 4 shows a filter arrangement 100, wherein a filter element 10 is accommodated in a frame 20. As already stated, the filter element 10 has a functional space 2. For the sake of greater clarity, sensor elements 6a and fragrancing elements 7 which may be present are not shown here. Since the functional space 2 is integrated in the filter element 10 and the external dimensions of the filter element 10 can remain unchanged despite the presence of the functional space 2, the filter element 10 can be easily mounted in frames 20 in existing machines and systems. Nor does the presence of the functional space 2 mean that additional sealing is needed between the filter element 10 and the frame 20. The additional functionalities of the filter element 10 which are created by the sensor elements 6a and the fragrancing elements 7 can therefore be obtained without modifying the system around the filter element 10, i.e. without alterations to the frame 20 and filter arrangement 100.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### LIST OF REFERENCE SIGNS

1 Bellows made of pleated filter medium
2 Functional space
3 Edge strips
4 Interface
5a Sensor element cutout
5b Fragrancing element cutout
6 Sensor
6a Sensor element
6b Sensor component
7 Fragrancing element
8 Transmitter
9 -
10 Filter element
20 Frame
100 Filter arrangement
a Raw gas side
b Clean gas side
L Air stream

What is claimed is:

1. A filter element, comprising:
a bellows comprising a pleated filter medium and edge strips bounding the bellows; and
a functional space that adjoins the bellows and is arranged within the edge strips, having an interface between the bellows and functional space,
wherein the interface is provided with at least one cutout configured to receive sensor elements and/or fragrancing elements.

2. The filter element according to claim 1, wherein at least two cutouts configured to receive at least one sensor element and at least one fragrancing element are provided in the interface.

3. The filter element according to claim 1, wherein at least two cutouts configured to receive at least two sensor elements are provided in the interface, and
wherein at least one cutout is positioned on a clean gas side of the bellows and at least one cutout is provided on a raw gas side of the bellows.

4. The filter element according to claim 1, wherein at least one cutout configured to receive at least one fragrancing element is provided in the interface,
wherein the at least one cutout is positioned on a clean gas side of the bellows, and/or
wherein at least one cutout configured to receive at least one fragrancing element is provided in the interface, the at least one cutout being positioned on a raw gas side of the bellows and the fragrancing element exhibits a metered substance having an oxidizing action, and/or
wherein at least one cutout configured to receive at least one fragrancing element is provided in the interface, the at least one cutout being positioned on a clean gas side of the bellows and the fragrancing element exhibits a metered substance having an oxidizing action.

5. The filter element according to claim 1, wherein the functional space is open only towards one side, the one side comprising a raw gas side of the bellows.

6. The filter element according to claim 1, wherein the functional space extends parallel to fold edges of the bellows, and
wherein fold surfaces of the bellows are penetrated by sensor elements and/or fragrancing elements.

7. The filter element according to claim 6, wherein the interface comprises filter medium or an edge strip.

8. The filter element according to claim 1, wherein the functional space is positioned in a manner adjoining end faces of fold edges of the bellows and the interface comprises an edge strip.

9. The filter element according to claim 1, wherein a sensor component is arranged in the functional space, and
wherein the sensor component and the sensor element form a sensor.

10. The filter element according to claim 9, wherein a transmitter is arranged in the functional space, the transmitter being connected to the sensor for data transmission of measurement data from the sensor.

11. A filter arrangement, comprising:
a frame; and
the filter element according to claim 1,
wherein the filter element is accommodated in the frame.

12. A method of using the filter arrangement according to claim 11 for air filtration in a mobile piece of equipment comprising a motor vehicle or commercial vehicle.

13. A method for controlling a filter system having the filter arrangement according to claim 11 and a control device configured to control the filter element, comprising:
connecting the control device to at least one sensor in the filter element for data transmission; and
controlling the filter element based on measured values from the sensor.
